(19) United States
(12) Patent Application Publication
(10) Pub. No.: US 2018/0332855 A1
(43) Pub. Date: Nov. 22, 2018

(54) COMPOSITION AND PROCESS FOR REMOVING AND PREVENTING MILDEW AND FUNGAL GROWTH

(71) Applicant: El Sayed Arafat, Leonardtown, MD (US)

(72) Inventor: El Sayed Arafat, Leonardtown, MD (US)

(73) Assignee: United States of America as represented by the Secretary of the Navy, Patuxent River, MD (US)

(21) Appl. No.: 15/597,739

(22) Filed: May 17, 2017

(51) Int. Cl. A01N 59/14; A61L 2/18; A01N 43/36; A01N 25/30; A01N 25/02

(52) U.S. Cl. CPC A01N 59/14; A61L 2/18; A01N 25/02; A01N 25/30; A01N 43/36

(57) ABSTRACT

Composition and process for removing and preventing mildew, mold and fungal growth on various surfaces comprising powder and/or granule chemicals, consisting essentially of inorganic and organic active ingredients corrosion inhibitors, surfactants, activators, buffer reagents with or without water.

### ORIGIN OF INVENTION

The invention described herein was made by employee(s) of the United States Government and may be manufactured and used by or for the Government for governmental purposes without the payment of any royalties thereon or therefor.

### BACKGROUND OF THE INVENTION

This invention relates to compositions and to a process for using said compositions for removing and preventing mildew, mold and fungal growth on various surfaces. In many geographical regions, the unwanted growth of mold and mildew is a serious problem. The rapid growth of mold and mildew on metal surfaces such as aluminum, titanium and alloys thereof is a serious problem in humid geographical areas.

It is well known that mildew growth on interior surfaces of aircraft, particularly military aircraft, has been a chronic problem, especially when these aircraft are operated in humid climates. Maintenance procedures to remove mildew from aircraft surfaces have historically required many man-hours. In addition, previously approved materials were often inadequate. The use of unauthorized cleaning solutions, such as household chlorine bleach (sodium hypochlorite), can induce corrosion damage to critical metallic surfaces. To address these problems, NAVAIR has developed a corrosion-inhibited mildew remover (Navclean) that meets all NAVAIR and AMCOM corrosion and mildew cleaning requirements. Therefore, there is a need DOD-wide for a safe, fast, corrosion-inhibited mildew remover kit where the cleaning solution can be easily mixed and applied in the field. This will benefit all existing rotorcraft and some fixed wing aircraft in the Navy, Marine Corps, Army, Air Force, Coast Guard and National Guard. The compatibility of the new invention with metals and non-metals has been studied and showed that the invention is safe to use on metals and non-metals. In addition, the efficacy-screening test of the invention was conducted on two types of mildew fungi and showed successful results.

Mold and mildew are fungi that grow on the surfaces of objects, within pores, and in deteriorated materials. They reproduce by means of tiny spores that are invisible to the naked eye and float through the air. They produce allergens that can cause allergic reactions, and in some cases, potentially toxic substances (mycotoxins). They can cause discoloration and odor problems, deteriorate materials, and lead to allergic reactions as well as other health problems in susceptible individuals. Numerous reports document fungal growth in passenger compartments, bilges and other areas of in-service aircraft. One study of fungal contamination on the interior surface of H-46 and H-53 helicopters at Fleet Readiness Center, Cherry Point, N.C., isolated eight different types of microfungi. The study also indicated that some corrosion of unprotected aluminum surfaces could be attributed to bacterial and fungal growth.

The current Navclean kit contains two ingredients in liquid form and two ingredients in dry forms that are packaged in four different containers. The liquid components of the current mildew remover kit cause difficulties in storage stability and transportation. Therefore, it is essential for DOD services to use a corrosion-inhibited mildew remover that can be safely stored and transported, then easily mixed and applied in the field. The new formulation is entirely dry, easier to transport, and has extended shelf life properties. In addition, the formulation will improve the shelf life, packaging preparation, storage and transportation.

The United States Environmental Protection Agency (EPA) under provisions of the Federal Insecticide, Fungicide and Rodenticide Act (FIFRA) currently registers pesticides. FIFRA requires that EPA register products only after registrants prove to the agency that the product will not pose unreasonable adverse effects as used. Further, each state must register products after they receive EPA registration and before they may be sold in any state. In addition, FIFRA requires that registration must be accompanied by its own nationally uniform EPA approved labeling. Labeling is the means by which EPA ensures that a pesticide product's manufacturer provides warnings and directions for use that are adequate to protect health and the environment.

Products that claim to kill any living organism, such as insects (insecticides), bacteria (disinfectants), algae (algaecides), molds and mildew (fungicides), weeds (herbicides) and so forth, must be registered with the EPA. Fungicide packaging labels must have an EPA registration number and instructions indicating the best way to reach, dispose and apply the registered product against targeted fungi. Without the EPA registration number on the package, no statements of product performance regarding the lowering of fungus counts or killing fungus on hard surfaces, sanitizing, germicidal, or disinfecting can be made.

### Summary of the Mildew Remover Invention

All ingredients of the invention (mildew remover dry kit) are made of stable solid materials (powder and/or granules), soluble in water, and commercially available as shown in Tables (1 and 2). The invention includes the following ingredients: Two Active Ingredients (one organic and one inorganic), two Corrosion Inhibitors (one organic and one inorganic), Non-ionic Surfactants, Anionic Surfactants, Active Ingredient Activator (organic) and two Buffer Reagents.

TABLE (1)
Composition of Formula #4

| | Parts by Weight | Percentage |
|---|---|---|
| Chemicals | Weight (g) | % |
| Active Ingredient | | |
| 1-Sodium Perborate | 25.0 | 50.5 |
| 2-Pyrrolidone | 0.0 to 30 | |
| Corrosion Inhibitors | | |
| 1-Sodium Metasilicate | 4.0 | 8.1 |
| 2-Benzotriazol | 3.0 | 6.1 |
| Surfactants | | |
| 1-Sodium Lauryl Sulfate | 4.0 | 8.1 |
| 2-IGEPAL 970 | 3.0 | 6.1 |
| Active Ingredient Activator | | |
| 1-Tetraacetylethylene- | 3.0 | 6.0 |
| diamine (TAED) | | |
| Buffer reagents | | |
| 1-Sodium Carbonate | 2.5 | 5.0 |
| 2-Sodium Bicarbonate | 5.0 | 10.1 |
| Water | One Liter | 100.0 |

TABLE (2)
Composition of Formula #5

| | Parts by Weight | |
|---|---|---|
| Chemicals | Weight (g) | Percentage % |
| Active Ingredients | | |
| 1-Sodium Perborate | 10.0 or 5.0-35 | 18.3 |
| 2-Peroxydel-30 | 20 or 10-30 | 36.7 |
| Corrosion Inhibitors | | |
| 1-Sodium Metasilicate | 4.0 or 2.0-35 | 7.3 |
| 2-Benzotriazol | 3.0 or 1.0-5.0 | 5.5 |
| Surfactants | | |
| 1-Sodium Lauryl Sulfate | 4.0 or 1.0-10 | 7.3 |
| 2-IGEPAL 970 | 3.0 or 1.0-10 | 5.5 |
| Active Ingredient Activator | | |
| 1-Tetraacetylethylene- | 3.0 or 1.0-5.0 | 5.5 |
| diamine (TAED) | | |
| Buffer Reagents | | |
| 1-Sodium Carbonate | 2.5 or 1.0-5.0 | 4.6 |
| 2-Sodium Bicarbonate | 5.0 or 1.0-10 | 9.2 |
| Water | One Liter or 0 to 120 parts | 100.0 |
| | | by weight |

Note:
Peroxydel-30: 2-Pyrrolidone
IGEPAL 970: Ethoxylated Dinonylphenol and Branched Nonylphenols

The compositions (Formulas 4 and 5) are dissolved in water and used within about 24 hours to remove mildew from aircraft surfaces. After 24 hours, the efficiency rapidly declines, and by 96 hours, the formula is comparable to water in its effectiveness. The cleaning procedure for removing mildew from aircraft surfaces is specifically described below:

Prior to applying the mildew remover, clean all affected surfaces to remove oil and dirt using standard aircraft cleaning materials and processes specified in NAVAIR 01-1A-509-2 and other applicable documents. Liberally apply the mildew remover to affected surfaces using clean cheesecloth, clean cotton rags, soft bristle brushes or any related authorized cleaning gear per NAVAIR 01-1A-509-2. Scrubbing may be required. Allow mildew remover to remain on the surface for about 5-15 minutes. Thoroughly rinse treated surfaces with fresh water to ensure no residue remains. Inspect surfaces for any remaining mildew. Black or brown mildew growth should be removed; however, residual mildew staining remaining on the paint is acceptable and may appear light brown and/or gray in color. Re-apply mildew remover as necessary followed by thorough rinsing. Inspect treated surfaces for detergent residue. If any indication of residue remains, rinse treated surfaces again with fresh water and re-inspect. Repeat the process as necessary to remove all residues.

The compatibility of the compositions with metals and non-metals were studied in accordance with the requirements of MIL-PRF-8557E specification. The following test methods were considered important for use of the invention as follows:
Sandwich Corrosion Test (ASTM F 1110-02)
Total Immersion Corrosion (ASTM F 483-02)
Hydrogen Embrittlement (ASTM F 519-97)
Cadmium Corrosion Test (ASTM F 1111-98)
Effect on Painted Surfaces (ASTM F 502-02)
Effect on Plastics (ASTM F 484-02)
Effect of Polyimide Wire (Section 4.5.11, MIL-PRF-85570E)
pH Value (ASTM E 70-02)

The compatibility test results for the compositions or formulations on metals and non-metals are shown in Table 3. The test results indicate that the formulation is safe to use on different metals and non-metals surfaces without causing any damage. Besides the use of the formulations on aircraft surfaces, the formulations could be used for removing mildew from other surfaces such as boats, ships and other industrial applications.

TABLE (3)
Test of Formula #5 in accordance with MIL-PRF-85570
(Cleaning Compound, Aircraft Exterior)

| | | | Mildew |
|---|---|---|---|
| | | | Remover |
| | | | Dry Kit |
| Requirements | Specification Limits | | Formulation |
| pH (ASTM E 70) | 7-10 | | 9.23 |
| Sandwich Corrosion | Not more than distilled water | | Pass |
| Test (ASTM F 110) | | | |
| Total Immersion | No Visible Corrosion | | |
| Corrosion Test | | | |
| (ASTM F 483) | | | |
| | | mg/cm²/day | mg/cm²/day |
| | Al 7075 (250/12) | 0.04 | 0.0002 |
| | Steel 1020 | 0.04 | 0.0000 |
| | (AMS 5046) | | |
| | Ti 6Al 4V | 0.04 | 0.0002 |
| | (AMS 9046) | | |
| | Mg AZ31B | 0.20 | 0.0004 |
| | (AMS4377) | | |
| Cadmium Corrosion | mg/cm²/day | 0.20 | 0.0004 |
| (ASTM F 1111) | | | |

| | | |
|---|---|---|
| Hydrogen | No Failure to 150 hr. when loaded | Pass |
| Embrittlement | at 45% | |
| (ASTM F 519 1a) | | |
| Effect on Plastics | Acrylic Type A No | Pass |
| (ASTM F 484) | Crazing - 8 hrs. | |
| | Acrylic Type C No Crazing - 8 hrs. | Pass |
| | Polycarbonate MIL-P-83310 - | Pass |
| | 2 hrs. | |
| Effect on Painted | No Softening >1 Pencil Hardness | Pass |
| Surfaces | | |
| (ASTM F 502) | | |
| Effect on Polyimide | No dielectric leakage | Pass |
| Wire | No visual effect greater than that | |
| | of distilled water | |

The initial efficacy test for the formulations was performed at Covance Laboratory. Two dry formulations (#4 and #5) were sent to Covance to perform the efficacy-screening test. The testing results indicate that both formulations are effective in removing two mildew fungi from hard surface as shown in Table 4. The ingredients of both formulations are identical (active and inert) with the exception of formulation #5, which contains one additional active ingredient (Peroxydel-30). It was noticed that formulation #5 dissolves faster and easier than formulation #4 in water. Miscibility of the dry kit in water is essential to prepare the cleaning solution. Based on this observation, formulation #5 was selected as the optimal formulation for the mildew remover dry kit. A detailed procedure for the efficacy test is described in the Covance Laboratory Report.

TABLE (4)
Efficacy Screening Test for the Mildew Remover Dry Formulation

| | | Number of tubes exhibiting | |
|---|---|---|---|
| | | confirmed growth/Total | |
| | Contact Time | number of tubes^{a} | |
| Test System | (Minutes) | Formulation #4 | Formulation #5 |
| Aspergilius niger | 10 | 0/8 | 0/8 |
| ATCC ® 6275 | | | |
| Trichophyton | 10 | 0/8 | 0/8 |
| Mentagrophytes | | | |
| ATCC ® 9533 | | | |

^{a}Tubes checked for turbidity and streaked to Neopeptone agar on Day 3 and Day 10 post-exposure to determine presence of growth.

The initial efficacy screening test results for the dry formulations are shown in Table 4. This test was conducted far screening purpose. Eight carriers were inoculated per test organism (Aspergillus Niger ATCC® 6275 and Trichophyton mentagrophytes ATCC® 9533) and showed no growth after 10 minutes exposure (contact time). The acceptance criterion for the screening test was that the test organism would be killed on all eight carriers. To meet the requirements of the actual EPA registration for an initial claim, the test substance would need to kill the test organism on 59 out of 60 carriers with each of three separate samples from three lots (one at least 60 days old) when run under Good Laboratory Practice (GLP) Guidelines. To make additional claims about the test substance, the test organism must be killed on 10 out of 10 carriers from at least two separate batches.

To meet the fleet need, it is essential to identify and validate effective, safe and environmentally friendly products for mildew remover applications. The formulations or compositions will be used to remove mildew from weapon systems across DOD maintenance facilities as alternatives to the use of unauthorized cleaning solutions. The use of unauthorized products such as household chlorine bleach (sodium hypochlorite) can induce corrosion damage to critical metallic surfaces. Providing qualified product to all levels of maintenance will avoid risk of use of improper materials, which may compromise performance, safety and health. In addition, the outcomes of this invention are improving the fleet readiness and pollution prevention onboard ships and complying with the current EPA regulations. The novel formulas work quicker than current alternatives, are safer and have the potential to be used for other cleaning application and in other markets. The novel formulations are entirely dry, easier to transport, and have extended shelf life properties. The formulations will be implemented by NAVAIR for removing mildew from aircraft during normal cleaning procedures. Implementation will be accompanied by issuing an authorization letter and Interim Rapid Action Change (IRAC) to 01-1A-509. NAVAIR's general series manual for Cleaning and Corrosion Control. In addition, a commercial source or supplier needs to be developed so material is available for NAVAIR uses as well as other militaries like AMCOM, the Air Force, Coast Guard, etc. The invention formulation will benefit all existing rotorcraft in the Navy, Marine Corps, Army, Air Force, Coast Guard and National Guard. Some fixed wing aircraft, which generate mildew, can also benefit.

While this invention has been described by a number of specific examples, it is obvious that there are other variations and modifications which can be made without departing from the spirit and scope of the invention as particularly set forth in the appended claims.

1. Composition for removing and preventing mildew and fungal growth comprising, in parts by weight,
(a) active ingredients consisting essentially of from about 5.0 to 35 parts of an inorganic perborate and from about 0.0 to 30 parts of a 2-pyrrolidone
(b) corrosion inhibitors consisting essentially of from about 2.0 to 35 parts of an inorganic silicate and from about 1.0 to 5.0 parts of a triazole,
(c) surfactants consisting essentially of from about 1.0 to 10 parts of an inorganic sulfate and from about 1.0 to 10 parts of a phenol,
(d) an activator consisting essentially of from about1.0 to 5.0 parts of a diamine,
(e) buffer reagents consisting essentially of from about 1.0 to 7.5 parts of carbonates,
(f) and from about 0.0 to 120 parts water.

2. The composition of claim 1 wherein the active ingredient is a metal perborate.

3. The composition of claim 1 wherein the silicate is metasilicate and the triazole is benzotriazole.

4. The composition of claim 1 wherein the inorganic sulfate is a metal sulfate.

5. The composition of claim 1 wherein the activator is tetraacetylethylene diamine.

6. The composition of claim 1 wherein the buffer reagents are a combination of mono and polycarbonates.

7. The composition of claim 1 wherein the water content about one liter.

8. The composition of claim 1 wherein water content is zero.

9. The composition of claim 1 wherein the pyrrolidone ranges from about 10 to 30 parts.

10. The composition of claim 1 wherein the perborate is 10 parts, the 2-pyrrolidone is 20 parts, the silicate is 4.0 parts, the triazole is a 3.0 parts, the sulfate is 4.0 parts, the phenol is 3.0 parts and the water is one liter.

11. Process for removing and preventing mildew and fungal growth from various surfaces, treating said surfaces with effective amounts of a composition consisting essentially of from about:
(a) active ingredients consisting essentially of from about 5.0 to 35 parts of an inorganic perborate and from about 0.0 to 30 parts of 2-pyrrolidone
(b) corrosion inhibitors consisting essentially of from about 2.0 to 35 parts of an inorganic silicate and from about 1.0 to 5.0 parts of a triazole,
(c) surfactants consisting essentially of from about 1.0 to 10 parts of an inorganic sulfate and from about 1.0 to 10 parts of a phenol,
(d) an activator consisting essentially of from about1.0 to 5.0 parts of a diamine,
(e) buffer reagents consisting essentially of from about 1.0 to 7.5 parts of carbonates,
(f) and from about 0.0 to 120 parts water,

12. The process of claim 11 wherein the active ingredient is a metal perborate.

13. The process of claim 11 wherein the silicate is metasilicate and the triazole is benzotriazole.

14. The process of claim 11 wherein the sulfate is a metal sulfate and the phenol is nonyl phenol.

15. The process of claim 11 wherein the water content of the composition is about one liter.

16. The process of claim 11 wherein the water content of the composition content is zero.

17. The process of claim 11 wherein the phenol is ethoxylated dinonyl phenol.

18. The process of claim 11 wherein the diamine is tetra acetylethylene diamine.
